(19) United States
(12) Patent Application Publication
(10) Pub. No.: US 2021/0230540 A1
(43) Pub. Date: Jul. 29, 2021

(54) UMBILICAL CORD LINING STEM CELLS AND CONDITIONED MEDIA THEREOF

(71) Applicant: Rafael Gonzalez, Yorba Linda, CA (US)

(72) Inventor: Rafael Gonzalez, Yorba Linda, CA (US)

(21) Appl. No.: 17/205,910

(22) Filed: Mar. 18, 2021

Related U.S. Application Data
(62) Division of application No. 15/248,629, filed on Aug. 26, 2016.
(60) Provisional application No. 62/213,537, filed on Sep. 2, 2015.

(51) Int. Cl. C12N 5/0735; C12N 5/0775

(52) U.S. Cl. CPC C12N 5/0606; C12N 2533/90; C12N 2501/2306; C12N 2501/115; C12N 2501/15; C12N 2501/22; C12N 2501/11; C12N 2501/17; C12N 2502/025; C12N 2501/23; C12N 2501/2303; C12N 2501/25; C12N 2501/2318; C12N 2501/13; C12N 2501/10; C12N 2501/125; C12N 2501/148; C12N 2501/165; C12N 2501/21; C12N 2501/2304; C12N 2501/2307; C12N 2501/2308; C12N 2533/54; C12N 5/0665

(57) ABSTRACT

The present disclosure relates to compositions of ULSCs and/or conditioned media thereof. In particular, the disclosure relates to conditioned media containing therapeutic factors including growth factors, cytokines, chemokines and extra cellular matrix components.

### PRIORITY

This application is a divisional of U.S. patent application Ser. No. 15/248,629, filed Aug. 26, 2016 which application claims the benefit of priority to U.S. Provisional Application No. 62/213,537 filed Sep. 2, 2015, whose disclosures are incorporated by reference in their entirety.

### FIELD

The disclosure relates to umbilical cord lining stem cells (ULSCs) and conditioned media thereof. The present disclosure relates to compositions of ULSCs derived from the lining of the umbilical cord and their conditioned media which contain therapeutic factors including growth factors, cytokines, chemokines and extracellular matrix (ECM) components.

### BACKGROUND

In the adult body there is constant loss and replacement of cells in the physiological process known as homeostasis. Homeostasis ensures that when cells are lost they are replaced by newly born cells derived from stem cells. Some tissues have stem cells that supply new cells at a fast rate like skin while others like the brain have slowly dividing stem cells capable of only limited cellular replacement. As we age replacement rates in all our tissues decrease because there are fewer surviving stem cells.

Adult bone marrow contains an important stem cell known as Mesencyhymal Stem Cells (MSCs). Fortunately MSCs are more prevalent in other tissues including fat, teeth and umbilical cords. MSCs are multipotent giving rise to a broad range of cell types but primarily form bone and connective tissue cells (Gonzalez et al. 2015, Pittenger et al. 1999). MSCs are useful in treating joint and connective tissue injuries where repair is often limited due to a lack of blood supply. When MSCs are transplanted into joints and other connective tissues they recruit blood vessels into the vicinity thereby enhancing repair.

A source of MSCs is-umbilical cords. Umbilical cords average 500 millimeters in length and 20 millimeters in diameter. The outer most cord tissue is known as the amniotic epithelium. It forms a tube which contains collagen fibers, fibroblast cells, and Wharton's jelly, (a gelatinous substance made largely from mucopolysaccharides and extracellular matrix) as well as two arteries, a vein and allantois duct. MSCs can be extracted from these structures having been found in cord blood, the vein, arteries, the amniotic epithelia, endothelium and Wharton's jelly (Subramanian et al. 2013).

The present disclosure relates to umbilical cord lining derived MSCs including but not limited to ULSCs isolated by the method described in U.S. Pat. No. 8,778,679. The aforementioned Patent and all related applications are incorporated herein by reference in their entirety.

### REFERENCES CITED

Gonzalez, R. et al. Stem Cells Targeting Inflammation as Potential Anti-aging Strategies and Therapies. Cell and Tissue Transplantation and Therapy. Libertas Academic. 2015:7.

DeMiguel, M. P. et al. Immunosuppressive properties of mesenchymal stem cells: advances and applications. Curr Mol Med. 2012; 12:574-591.

Calabro, A. et al. Fluorophore-assisted carbohydrate electrophoresis (FACE) of glycosaminoglycans. Journal of the Osteoarthritis Research Society International. 2001:9, Supp A: 16-22.

Calabro, A. et al. Microanalysis of enzyme digests of hyaluronan and chondroitin/dermatan sulfate by flurophore-assisted carbohydrate electrophoresis (FACE). Glycobiology: vol 10 no. 3, p. 273-281 (2000).

Cantinieaux, D. et al. Conditioned Medium from Bone Marrow-Derived Mesenchymal Stem Cells Improves Recovery after Spinal Cord Injury in Rats: An Original Strategy to Avoid Cell Transplantation. PLOS One. August 2013, Vol 8, Iss 8.

Tullberg-Reinert, H. et al. In situ measurement of collagen synthesis by human bone cells with a Sirius Red-based colorimetric microassay: Effects of transforming growth factor beta 2 and ascorbic acid 2-phosphate. Histochem Cell Biol (1999) 112:271-276.

Copelan, E. A. Hematopoietic Stem-Cell Transplantation. The New England Journal of Medicine. 2006: 354: 1813-26.

Ariff, B. et al. The Therapeutic Potential, Challenges and Future Clinical Directions of Stem Cells from the Wharton's Jelly of the Human Umbilical Cord. Stem Cell Reviews and Reports. April 2013, Volume 9, Issue 2, pp 226-240

Broxmeyer, H. et al. Human umbilical cord blood as a potential source of transplantable hematopoietic stem/progenitor cells. PNAS. 1989 May; 86(10):3828-32.

Mayani, H. A glance into somatic stem cell biology: basic principles, new concepts, and clinical relevance. Arch Med Res. 2003, 34:3-15.

Thomson, J. et al. Blastocysts Embryonic Stem Cell Lines Derived from Human. 1998. Science 282 (5391): 1145-1147.

Chambers, I. et al. Functional expression cloning of Nanog, a pluripotency sustaining factor in embryonic stem cells 2003, Cell, vol 113, no. 5, pp. 643-55.

Lu, L. L. et al. Isolation And Characterization Of Human Umbilical Cord Mesenchymal Stem Cells With Hematopoiesis-Supportive Function And Other Potentials Haematologica January 2006 91: 1017-1026.

Chen, G. et al. Human Umbilical Cord-Derived Mesenchymal Stem Cells Do Not Undergo Malignant Transformation during LongTerm Culturing in Serum-Free Medium, PLOS, June 2014, Volume 9, Issue 6, e98565.

Von Ah, D. et al. The Caregiver's Role Across the Bone Marrow Transplantation Trajectory. Cancer Nursing, Vol. 0, No. 0, 2015.

Subramanian, A. et al. Comparative Characterization of Cells from the Various Compartments of the Human Umbilical Cord Shows that the Wharton's Jelly Compartment Provides the Best Source of Clinically Utilizable Mesenchymal Stem Cells. PLOS One. Jun. 10, 2015.

Frisbie, D. D. et al. Clinical update on the use of mesenchymal stem cells in equine orthopaedics. Equine Veterinary Journal, 2010, 42: 86-89.

Lee, S. J. et al. High-resolution donor-recipient HLA matching contributes to the success of unrelated donor marrow transplantation. Blood December 2007, 110(13) 4576-4583.

Halme, D. G. et al. FDA Regulation of Stem-Cell-Based Therapies. N Engl J Med 2006; 355:1730-1735, Oct. 19, 2006.

Civin, C. I., et al. Antigenic analysis of hematopoiesis. III. A hematopoietic progenitor cell surface antigen defined by a monoclonal antibody raised against KG-1a cells. J Immunol 1984; 133:157-165.

Dominici M., et al. Minimal criteria for defining multipotent mesenchymal stromal cells. The International Society for Cellular Therapy position statement. Cytotherapy 8 (4): 315-317. 2006.

Pittenger M. F., et al. Multilineage potential of adult human mesenchymal stem cells. Science, 1999, Apr. 2; 284(5411):143-7.

Kern, S. et al. Comparative Analysis of Mesenchymal Stem Cells from Bone Marrow, Umbilical Cord Blood, or Adipose Tissue. STEM CELLS, 24: 1294-1301. 2006.

Shen, C. et al. Conditioned medium from umbilical cord mesenchymal stem cells induces migration and angiogenesis. Molecular Medicine Reports 12.1 (2015): 20-30.

Guan, L. et al. Therapeutic efficacy of umbilical cord-derived mesenchymal stem cells in patients with type 2 diabetes″. Experimental and Therapeutic Medicine 9.5 (2015): 1623-1630.

Tordeschi, M. R. et al. Transplanted Umbilical Cord Mesenchymal Stem Cells Modify the In Vivo Microenvironment Enhancing Angiogenesis and Leading to Bone Regeneration. Stem cells and development. Stem Cells Dev. 2015. Jul 1; 24(13):1570-81.

Wang K. X et al. The Effects of Secretion Factors from Umbilical Cord Derived Mesenchymal Stem Cells on Osteogenic Differentiation of Mesenchymal Stem Cells. PLoS ONE 10(3): 2015.

Kyurhchiev, D. et al. Secretion of immunoregulatory cytokines by mesenchymal stem cells. World Journal of Stem Cells. November 26; 6(5): 552-570. 2014.

### SUMMARY

The present disclosure provides compositions of ULSCs and their conditioned media.

Non-limiting examples of therapeutic factors present in ULSCs conditioned media are growth factors, cytokines, chemokines, and ECM components.

Non-limiting examples of growth factors which may be harvested from ULSC conditioned media and ULSC cells include but are not limited to Stem Cell Factor (SCF), Nerve Growth Factor (NGF),Vascular Endothelial Growth Factor (VEGF) family proteins, Epidermal Growth Factor (EGF) family proteins, Fibroblast Growth Factor (FGF) family proteins, Transforming Growth Factor beta (TGF-β) family proteins, Angiopoietin family proteins, Brain Derived Neurotrophic factor family proteins.

Non-limiting examples of cytokines which may be harvested from ULSC conditioned media and ULSC cells include but are not limited to Granulocyte Macrophage Colony Stimulating Factor (GM-CSF), Interferon Gamma (INF-γ), Interleukins (IL-3), (IL-4), (IL-6), (IL-7), (IL-8), (IL-10), (IL-17), (IL-18), and Tumor Necrosis Factor alpha (TNFα) family proteins. Other cytokines which may be present include the remaining interleukin family proteins and interferon family proteins.

Non-limiting examples of chemokines which may be harvested from ULSC conditioned media and ULSC cells include but are not limited to Macrophage Inflammatory Protein 1 beta (MIP-1β), Monocyte Chemotactic Protein 1 (MCP-1) and may include others from all four chemokine families including but not limited to C chemokines (XCL1 and XCL2), CC chemokines (CCL1-CCL28), CXC chemokines (CXCL1-CXCL17), CX3C chemokines (CX3CL1).

Non-limiting examples of ECM components which may be harvested from ULSC conditioned media and ULSC cells include but are not limited to Hyaluronic Acid (HA), Chondroitin Sulfate (CS), and Collagen. Other extracellular matrix constituents which may be present include Heparan sulfate, Keratin sulfate, elastin fibers, fibronectins and laminins.

### BRIEF DESCRIPTION OF THE DRAWINGS

FIG. 1. contains histograms of cytokines and chemokines in ULSC and BMSC conditioned media assayed by a quantitative multiplexed immunoassay system (MAPs).

FIG. 2. contains histograms of growth factor release in ULSC and BMSC conditioned media assayed by Enzyme Linked Immunosorbent Assay (ELISA).

FIG. 3. contains fluorophore-assisted carbohydrate electrophoresis (FACE) analysis demonstrating size and quantity of HA and CS in ULSC and BMSC conditioned media.

FIG. 4. contains an image of a hyaluronan size analysis by agarose gel electrophoresis.

FIG. 5. contains an image of a hyase digest size analysis by agarose gel electrophoresis.

FIG. 6. contains a histogram depicting levels of HA and CS in both conditioned media samples and the cells that produced said media.

FIG. 7. contains a data table and absorbance curve of Collagen in ULSC conditioned media assayed by dilution and absorbance analysis.

### DETAILED DESCRIPTION

Various embodiments of the invention are described in detail and may be further illustrated by the provided examples. As used in the description herein and throughout the claims that follow, the meaning of “a,” “an,” and “the” includes the plural reference unless the context clearly dictates otherwise. Also, as used in the description herein, the meaning of “in” includes “in” and “on” unless the context clearly dictates otherwise.

Throughout this specification and claims, the word “comprise,” or variations such as “comprises” or “comprising” will be understood to imply the inclusion of a stated integer or group of integers but not the exclusion of any other integer or group of integers.

Terms used in this specification generally have their ordinary meanings in the art, within the context of the invention, and in the specific context where each term is used. Certain terms that are used to describe the invention are discussed below, or elsewhere in the specification, to provide additional guidance to the practitioner in describing the compositions and methods of the invention and how to make and use them. For convenience, certain terms may be highlighted for example using italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms does not exclude the use of other synonyms. The use of examples anywhere in this specification, including examples of any terms discussed herein, is illustrative only, and in no way limits the scope of the invention so long as the data are processed, sampled, converted, or the like according to the invention without regard for any particular theory or scheme of action.

Further, whenever the name of a specific growth factor, cytokine, chemokine or extracellular matrix component is given, it is assumed to include all homologues, family members and splice variants of that factor which may also be present.

In all of the following examples which represent non-limiting preferred embodiments of the invention, pure ULSC cell lines obtained according to prior art methods (U.S. Pat. No. 8,778,679) and bone marrow mesenchymal stem cells (BMSC) lines are propagated based upon the following protocol. Culture vessels are seeded with cells at density of 1×10³ cells per cm² in Dulbecco's Modified Eagle Medium (DMEM) with low glucose and 15% fetal bovine serum (FBS), hereafter referred to as expansion media. Every 3 days the expansion media is replaced with fresh sterile expansion media. ULSCs are allowed to grow in a CO₂ incubator at 37° C. until they reach 80-90% confluency. Once confluent the media is replaced with sterile conditioning media consisting of 98% Roswell Park Memorial Institute Medium (RPMI), 0% FBS or alternately, 2% FBS, 1% Penicillin and Streptomycin, 1% Non Essential Amino Acids (NEAA), 1% Modified Eagle Medium (MEM), 1% Insulin, Transferrin, Selenium (ITS) supplement, 1% 100× Glutamine supplement, optionally Glutamax. In another embodiment, conditioning media consisting of 98% RPMI or an equivalent thereof, 0% FBS, 1% Glutamax. Conditioned media is collected at regular intervals into a sterile polyethylene terephthalate (PET) container that is stored at −20° C. and replaced with fresh sterile conditioning media. The process of collecting conditioned media by this method may be continued indefinitely.

Where applicable in the examples conditioned media may be purified and concentrated according to the following protocol. Collected conditioned media is thawed at 4° C. Collected lots are pooled and then filtered with a 0.22 micron (um) sterilizing filter. Filtered conditioned media is added to sample filter centrifugation cup which is then added to the Centricon Plus-70 filtrate collection cup and the combination is placed in a balanced centrifuge. The centrifuge is spun at up to 3500×g until a desired concentration is obtained, typically between 50-90 minutes. Concentrated media may then be transferred from the filtrate collection cup for further application as desired.

### EXAMPLE 1

### Cytokine and Chemokine Assay

Media from ULSCs, two sets from passage 3 and two sets from passage 6, in both expansion and conditioned media and four identical cultures of bone marrow mesenchymal stem cells BMSCs were assayed for the presence of cytokine and chemokine proteins by quantitative multiplexed immunoassay analysis (MAPs). The cytokines and chemokines profiled included: GM-CSF, INF-γ, IL-3, IL-4, IL-6, IL-7, IL-8, IL-18, MIP-1β, MCP-1, TNF-α. The MAPs analysis is based upon a capture-sandwich wherein capture antibodies are attached to fluorescently encoded microspheres. After capture of antigen from a biological sample such as cell culture media the antigen is detected and quantified using specific detection antibodies coupled to a fluorescent probe. Results obtained from this analysis were given as weight per volume of each analyte. The results were then normalized per number of cells from the corresponding well.

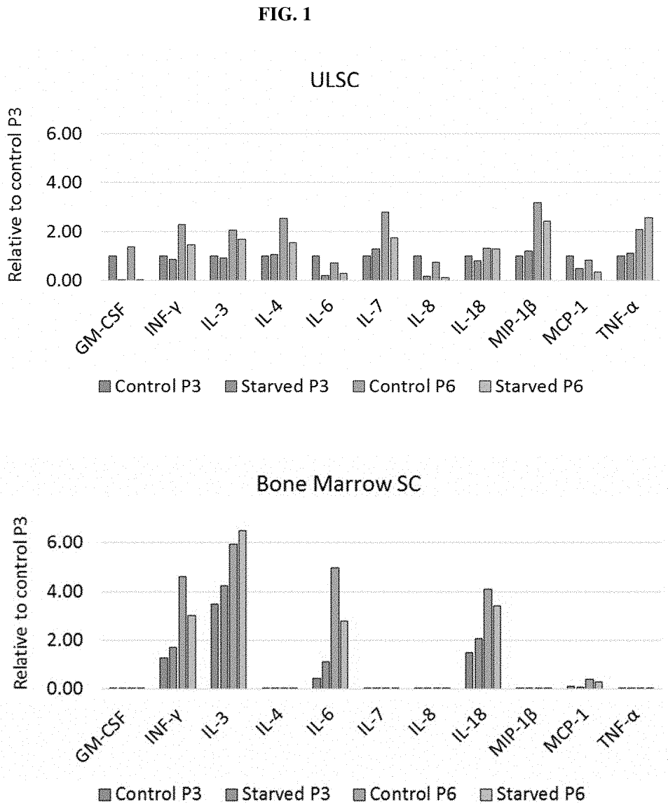

The results in FIG. 1 demonstrate that ULSCs express and secrete considerably more GM-CSF, IL-4, IL-7, IL-8, MIP-1β, MCP-1, and TNF-α than BMSCs. ULSCs express all the factors made by BMSCs and at least seven more factors not found at significant levels in BMSCs, These results are indicative but not exhaustive of the cytokines and chemokines which may be present in ULSCs conditioned media and cell culture tissue. Other cytokines which may be present include those from all four cytokine families: the four α-helix bundle family (the IL-2 subfamily, the interferon subfamily, the IL-10 subfamily), the IL-1 family (primarily IL-1 and IL-18), the IL-17 family and the cysteine-knot family (transforming growth factor beta superfamily including TGF-β1, TGF-β2, TGF-β3). Other chemokines which may be present include those from all four classes including C chemokines (XCL1 and XCL2), CC chemokines (CCL1-CCL28), CXC chemokines (CXCL1-CXCL17), CX3C chemokines (CX3CL1).

### EXAMPLE 2

### Human Growth Factor Assay

Conditioned media obtained from ULSCs and BMSCs was assayed for the presence of VEGF and SCF proteins by an Enzyme Linked Immunosorbent Assay (ELISA). Conditioned media was collected from a total of 12 cultures, two sets of ULSCs and two sets of BMSCs at 3, 6, and 9 days. Controls tested included 2% FBS media, 0% FBS media, 0% media plus vitamin B3, ULSCs in chondro media, and ULSCs in a transwell plate with a chondrocyte pellet. Concentrated conditioned media from each sample was added to each ELISA test strip well and incubated for one hour with gentle shaking. The ELISA strip was washed with assay buffer to stop the reaction and the optical density of each ELISA strip well was measured with a microplate reader at 450 nm.

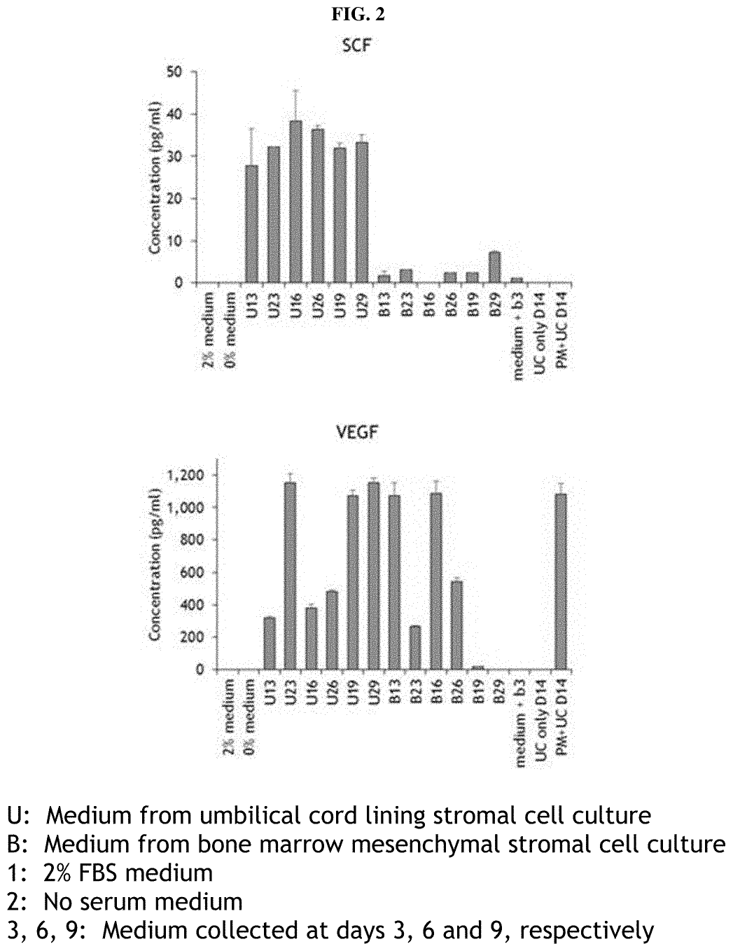

The results in FIG. 2 demonstrate that SCF is expressed and secreted at a considerably higher level in both sets of ULSCs at all time points compared to both sets of BMSCs. The results also show that both ULSCs and BMSCs express significant levels of VEGF but that after 9 days BMSCs secrete less VEGF than ULSCs. These non-limting examples are indicative but not exhaustive of the growth factors which may be present in ULSCs conditioned media and cell culture tissue. Other growth factors which may be present include VEGF family proteins, Epidermal Growth Factor (EGF) family proteins, Fibroblast Growth Factor (FGF) family proteins, Transforming Growth Factor beta (TGF-β) family proteins, Angiopoietin family proteins, Brain Derived Neurotrophic factor family proteins.

### EXAMPLE 3

### Hyaluronic Acid and Chondroitin Sulfate Assays

Media was collected from both ULSCs and BMSCs either daily or every third day from cell cultures propagated in either growth media or conditioning media. The cell layers in each well were also analyzed. The filtered and concentrated media is treated with proteases and nucleases to remove protein and nucleic acid, then treated with detergent and alcohol to remove lipids, leaving a mixture enriched with complex carbohydrates including glycosaminoglycans (GAGs). The GAGs mixture was then treated with a hyaluronidase enzyme which cleaves the GAGs into their constituent parts. The hyaluronic acid fragments were then separated by either polyacrylamide gel electrophoresis (PAGE) or agarose gel electrophoresis.

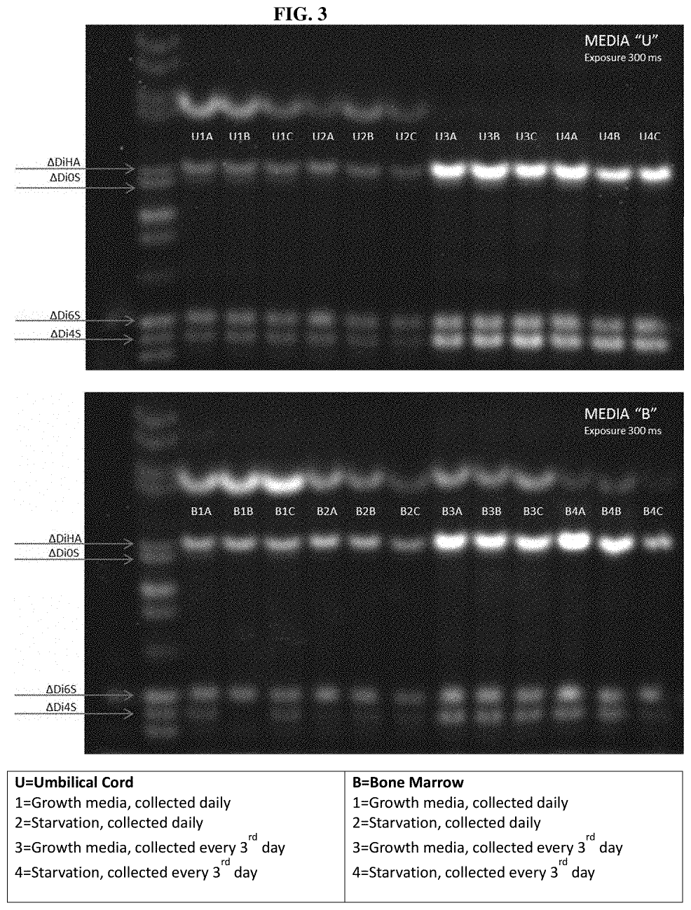

In FIG. 3, the cleaved GAGs were treated with mercuric ion, and then tagged by reductive amination giving an identical fluorescent signal for every free reducing group. The fluoro-tagged products were then separated by PAGE and scanned by CCD camera then analyzed with quantitative image analysis software. The results shown here demonstrate the presence of significant levels of HA and CS in the conditioned media of both ULSCs and BMSCs. These non-limiting examples are indicative but not exhaustive of the extracellular matrix constituents which may be present in ULSCs conditioned media and cell culture tissue. Other extracellular matrix constituents which may be present include: heparan sulfate, keratin sulfate, elastin fibers, fibronectins and laminins.

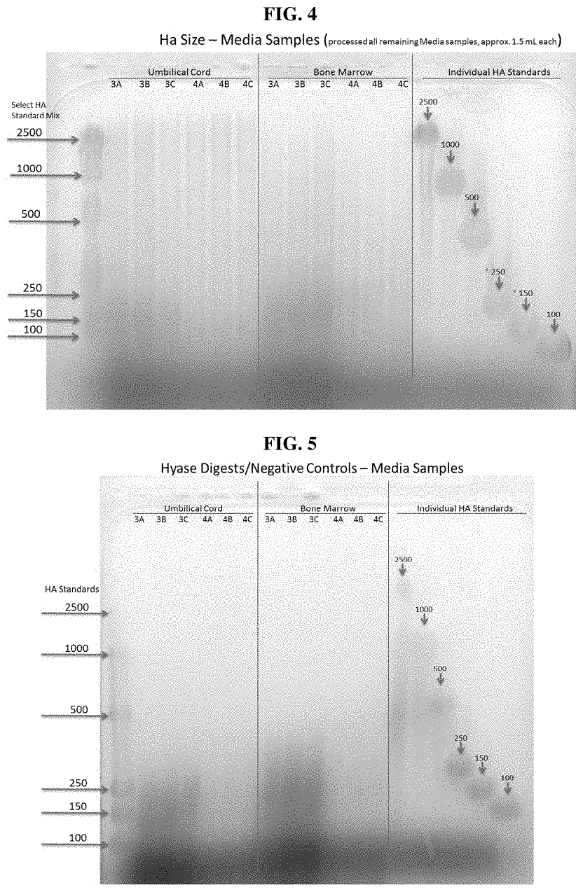

In FIG. 4 and FIG. 5, the cleaved GAGs were then separated on an agarose gel and stained for visualization prior to imaging. The results in FIG. 4 and FIG. 5 demonstrate that a considerable fraction of the HA present is in the media of both ULSC and BMSC cultures is high molecular weight up to at least 2500 kD. When further digested with Hyase (FIG. 5), HA accumulates in low molecular weight fragments at the bottom of the gel.

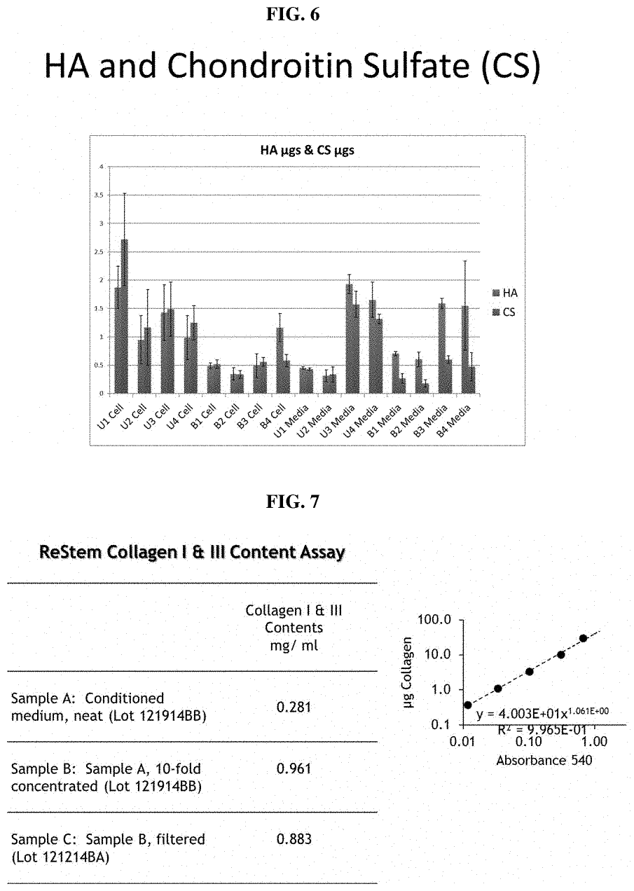

FIG. 6 provides a quantitation of the amounts of HA and CS present in both ULSC and BMSC cells and conditioned media. ULSC cells have increased quantities of both HA and CS compared to BMSCs and in conditioned media ULSCs produced more CS than BMSCs whereas HA levels were similar.

### EXAMPLE 4

### Collagen Assay

Collagen standards are prepared at 3⁰, 3⁻¹, 3⁻², 3⁻³ and 3⁻⁴ dilutions from 3 mg/ml stock in phosphate buffered saline. Ten microliters of ULSC conditioned media is used to make Collagen samples in triplicate. Sample A is cell culture medium supplemented with 2% fetal bovine serum and 1% ITS conditioned for 2 days by confluent ULSCs. Sample B is sample A concentrated 10 fold by centrifugation filter. Sample C is sample B filtered through 3MM Whatman paper. The test samples are deposited in a well of a 96-well cluster, and air-dried. Sample A is tested as 3⁰, and 3⁻¹ dilutions; samples B and C are tested as 3⁰, 3⁻¹, 3⁻², and 3⁻³ dilutions. One hundred microliters Bouin's fluid were added to each well and incubated at 37° C., for 1 hour, in a humidified enclosure. The Bouin's fluid was removed by aspiration, and the well was washed five times, each time with 200 μl deionized water, and air-dried. Seventy five microliters working Sirius Red solution were added to each well, and also to a set of 3 empty wells, serving as background controls, and incubated at 37° C., for 1 hour, in a humidified enclosure. The dye solution was removed by aspiration, and the well was washed five times, each time with 400 μl deionized water, and air-dried. One hundred microliters of 0.1 N NaOH were added to each well and agitated for 1.5 hours at ambient temperature. Absorbance at 540 nm (A 540) was determined using a microtiter plate reader. Standard collagen amounts were plotted vs. the average A-540 values to produce a standard curve with best fit equation of two variables. One average A-540 value of each conditioned medium sample was selected to substitute for the abscissa variable, to yield the ordinate value, which was corrected for the dilution factor and the volume of material used for testing (10 μl) to give the estimated collagen concentration in the original conditioned medium.

The results in FIG. 7 demonstrate that human Collagen is present at approximately 0.28 milligram/milliliter of ULSC conditioned media and may readily be concentrated to 1 mg/ml.

### EQUIVALENTS

Those skilled in the art will recognize, or be able to ascertain using no more than routine experimentation, many equivalents of the specific embodiments of the invention described herein. Such equivalents are intended to be encompassed by the following claims.

What is claimed is:

1. A composition of umbilical cord lining stem cells (ULSCs) and a conditioned media thereof comprising a Stem Cell Factor (SCF), a Vascular Endothelial Growth Factor (VEGF), a Granulocyte Macrophage Colony Stimulating Factor (GM-CSF), an Interleukin (IL) 4, 7, 8, a Tumor Necrosis Factor alpha (TNF-α), a Macrophage Inflammatory Protein (MIP) 1β, and a Monocyte Chemoattractant Protein (MCP) 1, Chondroitin Sulfate, Hyaluronic Acid and Collagen.

2. A concentrated composition of ULSCs conditioned media comprising:
a. a SCF from 20 to 50 picograms per milliliter (pg/ml); and
b. a VEGF from 200 to 1200 pg/ml; and
c. a cytokine selected from the group consisting of a GM-CSF, an IL4, 7, 8, a TNF-α at levels up to 4 times greater than in bone marrow mesenchymal stem cells (BMSCs) conditioned media; and
d. a chemokine comprising a MIP-1β, and a MCP-1 at levels up to 4 times greater than in BMSCs conditioned media; and
e. an extracellular matrix (ECM) component comprising a collagen I, a collagen III, a chondroitin sulfate (CS), and a hyaluronic acid (HA) at levels comparable to or greater than in BMSCs conditioned media; and
f. said HA being of high molecular weight up to at least 2500 kilodaltons (kd); and
g. said collagen I and said collagen III from 0.75 to 1 milligrams per milliliter (mg/ml).

3. A concentrated and sterilized composition of ULSCs derived conditioned media which comprises:
a. a SCF from 20 to 50 pg/ml; and
b. a VEGF from 200 to 1200 pg/ml; and
c. a cytokine selected from the group consisting of a GM-CSF, an IL4, 7, 8, a TNF-α at levels up to 4 times greater than in BMSCs conditioned media; and
d. a chemokine comprising a MIP-1β, and a MCP-1 at levels up to 4 times greater than in BMSCs conditioned media; and
e. an extracellular matrix (ECM) component comprising a collagen I, a collagen III, a chondroitin sulfate (CS), and a hyaluronic acid (HA) at levels comparable to or greater than in BMSCs conditioned media; and
f. said HA being of high molecular weight up to at least 2500 kilodaltons (kd);
g. said collagen I and said collagen III from 0.75 to 1 milligrams per milliliter (mg/ml).
